(19) United States
(12) Patent Application Publication
(10) Pub. No.: US 2010/0247667 A1
(43) Pub. Date: Sep. 30, 2010

(54) MATERIAL FOR SOFT TISSUE ENLARGEMENT

(75) Inventors: Takako Ariga, Ashigarakami-gun (JP); Takao Anzai, Ashigarakami-gun (JP); Jun Konishi, Ashigarakami-gun (JP)

Correspondence Address: BUCHANAN, INGERSOLL & ROONEY PC, POST OFFICE BOX 1404, ALEXANDRIA, VA, 22313-1404, US

(73) Assignee: TERUMO KABUSHIKI KAISHA, Shibuya-ku (JP)

(21) Appl. No.: 12/732,491

(22) Filed: Mar. 26, 2010

(30) Foreign Application Priority Data
Mar. 26, 2009 (JP) 2009-075689

(51) Int. Cl. A61K 9/50

(52) U.S. Cl. 424/501

(57) ABSTRACT

A soft tissue enlarging material including fine particles of a pH-response water-absorbing swelling polymer having an average particle size of from 15 μm to 40 μm wherein swelling of said particles is completed within 10 minutes after immersion in a 10 mM phosphate buffer physiological saline solution (pH: 7) at 37° C.

### TECHNICAL FIELD

An exemplary embodiment relates to a material for soft tissue enlargement adapted for use in the treatment, remedy and/or cure of soft tissues involved such as, for example, in urinary incontinence or vesicoureteric reflux.

### BACKGROUND

For a cure needing soft tissue enlargement, there is known a curing method wherein an interstitial agent is injected against patients of urinary incontinence or vesicoureteric reflux around the urethra or in the vicinity of the ureter. In the past, although an injection agent made up of polytetrafluoroethylene (PTFE) was investigated (see Berg, S., “Polytef augmentation urethroplasty; correction of surgically incurable urinary incontinence by injection technique”, Arch. Surg., 107(3):379-81 (September 1973), such an injection agent is made of a paste mixture made of fine particles of PTFE and a glycerine fluid. When a certain time passes after injection into a living body, the glycerine dissipates into the living body and is subjected to metabolism. The fine particles of PTFE remain as they are without undergoing hydrolysis and the like in the living body and are regarded as causing a problem such as of pulmonary obstruction and the like after migration to other sites of the body such as the lung, brain and the like.

It has been accepted that the migration of such fine particles to many other organs depends on the particle size and that the probability of occurrence is high when the size is not larger than 40 μm. For injection therapy, an attempt has been made to use a silicone suspended in a hydrogel. In this connection, however, it is considered that the fine particles of the silicone are possibly delocalized after migration to other organs via macrophages. In view of these, both the fine particles of PTFE and the silicone injection have safety concern.

On the other hand, there have been developed medical products wherein the size of fine particles is made larger than 40 μm to prevent phagocytosis by and migration to other organs with macrophages. Nevertheless, a larger particle size necessitates a larger needle used for injection, resulting in great invasiveness on the part of patient (e.g., pain).

Living body-derived materials have been studied, of which there is used an injection agent making use of collagen that is a naturally occurring polymer (see Japanese Patent Laid-open No. 2005-193055). The collagen injection agent is slow in tissue reaction within a living body and exhibits good affinity therefor and thus, the above-stated problem is significantly overcome. However, because of the rapid absorption of collagen, a difficulty is involved in maintaining the curing effect thereof. In order to prolong the absorption time in the body, a crosslinking agent such as glutaraldehyde can become necessary, but a toxic problem of residual glutaraldehyde exists at present.

### SUMMARY

Accordingly to an exemplary aspect, fine particles made of a synthetic polymer material that is capable of ameliorating or overcoming the above-stated problems and does not give a feeling of a foreign body in a living body and that is capable of being injected into a living body without migration to other organs, is provided.

According to another exemplary aspect, as fine particles for body injection, spherical fine particles that are easy for injection into a living body and exhibit small degrees of foreign body-reaction and inflammatory reaction at transplant sites injected therewith into the living body, are provided.

According to another exemplary aspect, a material for soft tissue enlargement is provided. Exemplary aspects of a soft tissue enlargement, for example, include:

(1) A soft tissue enlarging material, which consists essentially of fine particles of a pH-response water-absorbing swelling polymer having an average particle size of from 15 μm to 40 μm wherein swelling of said particles is completed within 10 minutes after immersion in a 10 mM phosphate buffer physiological saline solution (pH: 7) at 37° C.
(2) The soft tissue enlarging material as recited in (1) above, wherein the particle size is increased to 2 to 4 times greater than the original one by swelling in the physiological saline solution.
(3) A soft tissue enlarging material, which consists essentially of fine particles of a pH-response, water-absorbing swelling polymer having an average particle size of from 15 μm to 40 μm wherein swelling of the particles in a body fluid in a living body is completed within 10 minutes.
(4) The soft tissue enlarging material as recited in (1) or (3) above wherein the material is employed under the skin or beneath mucous membrane.

According another exemplary aspect, there can be provided fine particles, which can be injected by means of a finer syringe needle and are easy for injection, and involve a small risk of migration to other organs through foreign body reaction after injection and can be safely, reliably used for recovery and remedy of functions for soft tissue enlargement such as, for example, urinary incontinence or vesicoureteric reflux, or used as a bone repairing material. Thus, the fine particles are adapted for use as a material for soft tissue enlargement.

### DETAILED DESCRIPTION

In order to reduce the foreign body reaction, in a living body, of a material per se implanted under the skin or beneath the mucous membrane and migration to other organs and also to enable the material to be injected by means of a finer syringe needle, we have made intensive studies on particulate materials under the following exemplary concept. When a particulate material whose size allows injection with a syringe needle instantaneously absorbs a biogenic substance in a living body thereby permitting it to be swollen to such a size that is unlikely to undergo phagocytosis by inflammatory cells having the phagocytic function such as macrophages or neutrophils, it can become difficult to allow the material to be migrated to other organs. As a result of the studies, it has been found that fine particles of a pH-response, water-absorbing swelling polymer having a particle size of from 15 μm to 40 μm prior to swelling can be injected with a small syringe needle of not greater than 24 G and instantaneously absorb the fluid, etc. in a living body after injection and can be immediately swollen to larger-sized ones. This, for example, can lead to a very small risk involved in the migration to organs.

While not wishing to be bound by any particular theory, we have also found that probably because the particulate material per se exhibits characteristics close to those of biogenic substances by instantaneously absorbing the fluid, etc. in a living body after injection under the skin or beneath the mucous membrane, the material is, for example, unlikely to undergo a foreign body recognition reaction of inflammatory cells, so that tissue reactions occur very rarely. This can be very useful as a material to be implanted under the skin or beneath the mucous membrane.

Exemplary aspects are described in detail by way of typical, non-limiting examples.

The fine particles of a pH-response, water-absorbing swelling polymer can be made of a hydrogel. For the preparation, a monomer solution containing a monomer, a crosslinking agent, a pore-forming agent and a solvent can be used. An exemplary concentration of the monomer in the solvent is within a range of 20 to 30 w/w %. The fine particles can be prepared according to a reverse phase suspension polymerization technique.

The monomer used can include an ethylenically unsaturated monomer. In an exemplary embodiment, at least a part, for example, 10 to 15% and more preferably 10 to 30% of the monomer can be at least one member selected from acrylic acid, methacrylic acid and derivatives thereof. The monomers usable in combination with the monomers having these carboxylic moieties can be selected from monomers having relatively excellent mechanical characteristics, for which acrylamide and (meth)acrylamide monomers (a1) may be used without limitation. Specific examples include (meth)acrylamide, N-methyl(meth)acrylamide, N-ethyl(meth)acrylamide, N-n-propyl(meth)acrylamide, N-isopropyl(meth)acrylamide, N-n-butyl(meth)acrylamide, N-isobutyl(meth)acrylamide, N-s-butyl(meth)acrylamide, N-t-butyl(meth)acrylamide, N,N-dimethyl(meth)acrylamide, N-ethyl-N-methyl(meth)acrylamide, N,N-diethyl(meth)acrylamide, N-methyl-N-isopropyl(meth)acrylamide, N-methyl-N-n-propyl(meth)acrylamide, N-ethyl-N-isopropyl(meth)acrylamide, N-ethyl-N-n-propyl(meth)acrylamide, N,N-di-n-propyl(meth)acrylamide, diacetone(meth)acrylamide and the like. These (meth)acrylamide monomers (a1) can be used singly or in combination of two or more.

The crosslinking agent that is used can be any of polyfunctional ethylenically unsaturated compounds. N,N-methylenebisacrylamide can be preferred as the agent. An exemplary concentration of the crosslinking agent in the solvent is within a range of smaller than 1 w/w %, more preferably smaller than 0.1 w/w %.

The pore-forming agent can be one that is able to impart porosity to the fine particles. The impartment of porosity can be attained by creating a supersaturated suspension of a pore-forming agent in a monomer solution. Sodium chloride that is insoluble in the monomer solution and is soluble in a wash solution can be used. Alternatively, potassium chloride, iced water, sucrose, sodium bicarbonate and the like can be used. The particle size of the pore-forming agent can be smaller than 10 μm and, preferably smaller than 5 μm. A smaller particle size can, for example, better promote suspension of a pore-forming agent in the solution. An exemplary concentration of the pore-forming agent is within a range of 5 to 50 w/w %, more preferably 10 to 20 w/w %.

The solvent of the monomer solution can be selected depending on the types of monomer and crosslinking agent, the solubility of pore-forming agent and the continuous phase used for reverse phase suspension polymerization. An exemplary solvent is water and an exemplary concentration thereof is in the range of 20 to 80 w/w %, more preferably 50 to 80 w/w %.

As the continuous phase used for the reverse phase suspension polymerization, liquid paraffin, cyclohexane, toluene or the like can be used, of which liquid paraffin can be preferred because the dispersed state of a monomer solution can be held better if the specific gravity of the continuous phase is closer to the specific gravity of a solvent of the monomer solution.

The crosslinking density can substantially affect mechanical characteristics of the fine particles. The crosslinking density (and mechanical characteristics ascribed thereto) can be best controlled by changing the concentrations of monomer, crosslinking agent and solvent. Although the monomer can be crosslinked by oxidation-reduction or by application of a radiation ray or heat, a preferred type of crosslinking initiator is one which acts through oxidation-reduction and can include, for example, ammonium persulfate and N,N,N′,N′-tetramethylethylenediamine. After completion of polymerization, the resulting fine particles can be washed with water, an alcohol or an appropriate detergent solution, thereby removing the pore-forming agent, an unreacted residual monomer and an oligomer not taken therein.

The swelling rate of the fine particles can be controlled by subjecting ionic polyfunctional groups existing on the hydrogel network structure to protonation/deprotonation. A hydrogel can be prepared, from which excesses of the monomer and pore-forming agent can be rinsed away, followed by the step of controlling the swelling rate. In an embodiment wherein a pH-sensitive monomer having a carboxylate group is incorporated into the hydrogel network structure, the hydrogel can be incubated in a low pH solution. The resulting free protons in the solution can act to protonate the carboxylate on the hydrogel network structure. The duration and temperature of the incubation and the pH of the solution can influence a degree of control of the swelling rate. In general, the duration and temperature of the incubation can be directly proportional to the degree of swelling control and can be inversely proportional to the pH of solution. After completion of the incubation, an excess of the treating solution can be rinsed off from the hydrogel material and the material can be dried. When the hydrogel treated with the low pH solution is dried, it can become smaller in size than a non-treated hydrogel and can be injected with a syringe needle with a smaller inner diameter.

When a pH-sensitive monomer having an amine group is incorporated into the hydrogel network structure, the hydrogel can be incubated in a high pH solution. Under high pH conditions, deprotonation can occur on the amine group of the hydrogel network structure. The duration and temperature of the incubation and the pH of the solution can influence the degree of control of the swelling rate. Generally, the duration and temperature of the incubation and the pH of the solution can be directly proportional to the degree of swelling control. After completion of the incubation, an excess of the treating solution can be rinsed off from the hydrogel material, followed by drying.

The fine particles can be subjected to sieve classification in dried state after preparation by a reverse phase suspension polymerization technique thereby obtaining particles having a size within a desired range.

The particle size of the dried fine particles can range, for example, from 15 μm to 40 μm, preferably from 20 μm to 35 μm and more preferably from 25 μm to 30 μm. After immersion in a 10 mM phosphate buffer physiological saline solution (pH: 7) at 37° C., the fine particles can be swollen to a size of 2.0 to 5.0 times, preferably 2.5 to 4.5 times and more preferably 2.7 to 4.0 times, greater than the original ones.

### Example 1

An example of fine particles (with a dried particle size of 20 μm) of a pH-response, water-absorbing swelling polymer prepared, as a soft tissue enlarging material, according to a reverse phase suspension polymerization process is illustrated below.

Initially, 75 g of cyclohexane, 75 g of liquid paraffin and 2.0 g of sorbitan sesquioleate, all placed in a 300 ml of beaker, were agitated with a magnetic stirrer thereby preparing a continuous phase of reverse phase suspension polymerization. Further, a stream of nitrogen was passed for 30 minutes to remove dissolved oxygen from the continuous phase. On the other hand, 3.8 g of acrylamide, 2.2 g of sodium acrylate, 0.013 g of N,N-methylenebisacrylamide and 6.09 g of sodium chloride were weighed and placed in a 50 ml brown glass bottle, to which 19.9 g of distilled water was added, followed by dissolution under agitation with a magnetic stirrer to prepare an aqueous monomer solution.

Next, a solution of 0.27 g of ammonium persulfate dissolved in 2.0 g of distilled water was added to the aqueous monomer solution, all of which was added to the continuous phase solvent. The mixture was agitated by means of a mechanical stirrer rotated at a frequency of 200 r.p.m., thereby dispersing the monomer solution in the continuous phase solvent. After agitation for 30 minutes, the temperature was raised to 40° C., followed by further addition of 500 μl of N,N,N′,N′-tetramethylethylenediamine. Agitation was continued for 1 hour, after which the content of the beaker was transferred into 1 liter of dimethylsulfoxide. The resulting precipitate was collected on a filter paper, followed by washing with ethanol and hexanol and drying under reduced pressure. 2.5N hydrochloric acid was added to the precipitate and was allowed to stand in an oven at 55° C. for 24 hours. The acid-treated product was transferred into distilled water, followed by changing distilled water until no pH change of distilled water was observed. The product obtained after the washing was placed in ethanol and disintegrated by means of a magnetic stirrer, followed by classification with a stainless steel sieve (with a sieve mesh size of 25 μm) to obtain fine particles with an average particle size of 20 μm. It will be noted that the average particle size of the fine particles was measured by a Coulter counter (Model LS230, made by Beckman Inc.) in such a state that the fine particles were immersed in ethanol. Hereinafter, as average particle size prior to swelling, measurement was carried out in the same way.

### Example 2

The sample prepared in Example 1 and prior to the classification was classified with a stainless steel sieve (a fraction capable of passing through a sieve with a mesh size of 40 μm and left after passing through a sieve with a mesh size of 25 μm), thereby obtaining fine particles with an average particle size of 34 μm.

### Comparative Example 1

A comparative example of fine particles (with a dried particle size of 150 μm) of a pH-response, hydrous swelling polymer was prepared according to a reverse suspension polymerization technique for a soft tissue enlarging material.

Initially, 75 g of cyclohexane, 75 g of liquid paraffin and 2.0 g of sorbitan sesquioleate, all placed in a 300 ml of beaker, were agitated with a magnetic stirrer thereby preparing a continuous phase of reverse phase suspension polymerization. Further, a stream of nitrogen was passed for 30 minutes to remove dissolved oxygen. On the other hand, 3.8 g of acrylamide, 2.2 g of sodium acrylate, 0.013 g of N,N-methylenebisacrylamide and 6.09 g of sodium chloride were weighed and placed in a 50 ml brown glass bottle, to which 19.9 g of distilled water was added, followed by dissolution under agitation with a magnetic stirrer to prepare an aqueous monomer solution.

Next, a solution of 0.27 g of ammonium persulfate dissolved in 2.0 g of distilled water was added to the aqueous monomer solution, all of which was added to the continuous phase solvent. The mixture was agitated by means of a mechanical stirrer rotated at a frequency of 100 r.p.m., thereby dispersing the monomer solution in the continuous phase solvent. After agitation for 30 minutes, the temperature was raised to 40° C., followed by further addition of 500 μl of N,N,N′,N′-tetramethylethylenediamine. Agitation was continued for 1 hour, after which the content of the beaker was transferred into 1 liter of dimethylsulfoxide. The resulting precipitate was collected on a filter paper, followed by washing with ethanol and hexanol and drying under reduced pressure. 2.5N hydrochloric acid was added to the precipitate and was allowed to stand in an oven at 55° C. for 24 hours. The acid-treated product was transferred into distilled water, followed by changing distilled water until no pH change of distilled water was observed. The product obtained after the washing was placed in ethanol and disintegrated by means of a magnetic stirrer, followed by classification with a stainless steel sieve to obtain fine particles with an average particle size of 150 μm (i.e. a fraction capable of passing through a sieve with a mesh size of 500 μm and left after passage through a sieve with a mesh size of 100 μm).

### Comparative Example 2

The fine particles (with an average particle size of 150 μm) obtained in Comparative Example 1 were placed in a 10 mM phosphate buffer physiological saline solution (pH: 7) for 72 hours and were rendered hydrous and swollen.

### Test Example 1

### Implantation Test Under the Skin of Rat

A test example is illustrated below wherein the fine particles of the examples and the comparative examples were histologically assessed by use of SD rats in in vivo experiments. The implantation test of the fine particles under the skin was conducted in such a way that 3 mg of fine particles were implanted under the back skin of rats under anesthesia. After a given time of period, the rats were killed under carbonic acid gas and autopsied. Thereafter, the specimen and surrounding tissues were fixed in a 10% neutral buffer formalin solution, subjected to paraffin embedding and cut into thin slices by means of a microtome to obtained tissue pieces. The thus obtained thin pieces were dyed by hematoxylin-eosin staining and observed under optical microscope.

According to the results of this test, the following assessment is made. The material implanted in a living body serves as a foreign body, for which there are caused an inflammatory reaction ascribed to inflammatory cells such as granulocytes (e.g., neutrophils, eosinophils), lymphocytes, macrophages and the like. Accordingly, how granulocytes (e.g. neutrophils, eosinophils), lymphocytes and macrophages behave is observed according to the implantation test wherein the biocompatibility of the material can be judged. Granulocytes can have high migration properties and the phagocytic function and can appear at an initial stage of the inflammatory reaction, followed by reaction against the treatment of necrotic tissue, infection with microorganism such as bacteria, and foreign bodies. Macrophages can also have the active phagocytic function and detoxify, or digest or decompose harmful substances. Lymphocytes can have no phagocytic function but can play a leading part against an inflammation against viral infection or also against a chronic inflammation. In the implantation test, since the granulocytes and macrophages have the phagocytic function, they can act to phagocytize the implantation material. Although the lymphocytes have no phagocytic function, they can appear more frequently when the biocompatibility of an implantation material is lower, thus being adapted for judging a degree of the biocompatibility of material. In addition, a great amount of inflammatory cells can lead to local occurrence of necrosis and the like, with the possibility of causing migration of other inflammatory cells.

The results of observation under an optical microscope are shown in Table 1 below. In Table 1, the symbols reflect the following:

±—observed few cells or a low level
+—observed (more than ±)+
+—observed many cells or at a higher level (more than +)+
++—observed a great number of cells or a much higher level (more than ++)
++++—observed an extremely large number of cells or an extremely high level (more than +++)

TABLE 1
Results of pathological assessment of tissues

| | 7 days after implantation | | | | 28 days after implantation | | | |
|---|---|---|---|---|---|---|---|---|
| | | | Comp. | Comp. | | | Comp. | Comp. |
| | Ex. 1 | Ex. 2 | Ex. 1 | Ex. 2 | Ex. 1 | Ex. 2 | Ex. 1 | Ex. 2 |
| Phagocyte | ± | ± | + + + | + + | ± | ± | + + | + + |
| | | | | + + | | | | |
| Lymphocyte | + | + | + + + | + + | ± | ± | + + | + + |
| | | | | + + | | | | |
| Phagocytic | ± | ± | + + + | + + | ± | ± | + + | + + |
| level | | | | + + | | | | |

It is known that a smaller size of an implantation material pathologically can lead to a more vigorous degree of inflammatory reaction. However, Example 1 (average size of 25 μm prior to swelling) and Example 2 (average size of 34 μm prior to swelling) are weaker in inflammatory reaction than Comparative Example 1 (average size of 150 μm prior to swelling). With respect to the phagocytic cells (cells, such as granulocytes, macrophages and the like, having the phagocytic function), there were not observed, seven days after the implantation, phagocytic cells other than those appearing for a response reaction against an implanted material serving as a foreign body and implanted in the living body in Examples 1 and 2. Therefore, little phagocytosis of the implanted fine particles was observed. In contrast, Comparative Example 1 was such that an amount of phagocytic cells increased. Not only the increase in amount of the cells, but also a number of macrophages that phagocytized the implanted fine particles from therearound and were converted to foam cells were observed. With respect to the lymphocytes, no lymphocytes other than those appearing for the response reaction against the implanted materials of both Examples 1 and 2 were observed.

In contrast, in Comparative Example 1, the amount of lymphocytes apparently increased, thereby permitting a wide area to be wetted. 28 days after the implantation, the inflammatory reactions settled down. As to Comparative Example 1, phagocytic cells were present in a great number and foamy macrophages were observed. In Examples 1 and 2, phagocytic cells and lymphocytes were reduced in number and no foamy macrophages were observed.

In Examples 1 and 2, the fine particles in dry state were implanted, after which they were swollen while absorbing the body fluid in the living body. Thus, it is assumed that since most of the fine particles are made up of the living body-derived substance of their own, biocompatibility is enhanced. In Comparative Example 1, although implantation was made in dry state, the inflammatory reactions were more intense than those of the Examples 1, 2 seven days after the implantation. In Comparative Example 2 (average particle size of 150 μm prior to swelling), the fine particles being swollen with a 10 mM phosphate buffer physiological saline solution (pH: 7) were implanted. Seven days after the implantation, inflammatory reactions occurred much more intensely than with the cases of Examples 1, 2 and Comparative Example 1. Comparison with Comparative Example 1 using the same particle size revealed that greater numbers of phagocytic cells and lymphocytes apparently existed and because of the existence of a great number of phagocytic cells, phagocytosis against the fine particles became violent. After passage of 28 days after the implantation, exchange between the phosphate buffer solution (pH: 7) and the body fluid in the implantation material was probably completed, for which the numbers of phagocytic cells and lymphocytes and the phagocytic action on the fine particles settled down to such a level as in Comparative Example 1.

### Test Example 2

Change with time of swollen state 50 mg of the fine particles of each of Examples 1 to 3 were placed in 5 ml of a 10 mM phosphate buffer physiological saline solution (PBS) (pH: 7) and the time change of the particles was observed. Images were taken with a CCD camera and 50 particles were randomly chosen to measure particle sizes thereof, from which an average particle size was calculated. The results are shown in Table 2.

TABLE 2
Change with time of swollen state

| | Particle size in | Immediately after | | | | |
|---|---|---|---|---|---|---|
| | distilled water | immersion | After 10 | After 30 | After 90 | After 24 |
| | (before swelling) | in PBS | minutes | minutes | minutes | hours |
| Example 1 | 20 μm | 52 μm | 55 μm | 55 μm | 55 μm | 55 μm |
| Example 2 | 34 μm | 102 μm | 106 μm | 106 μm | 106 μm | 106 μm |
| Comparative | 150 μm | 184 μm | 190 μm | 216 μm | 250 μm | 270 μm |
| Example 1 | | | | | | |

From Table 2, it will be seen that in Comparative Example 1, swelling is not completed 10 minutes after the immersion in PBS. The detailed reason for this is not known at present. In Comparative Example 1, the implantation was conducted in dry state. When the results that the inflammatory reaction were more intense than in Examples 1, 2 seven days after the implantation are taken into account, the completion of swelling after passage of 10 minutes after immersion in PBS is considered to be important for the soft tissue enlarging material.

The detailed description above describes various aspects of a soft tissue enlarging material. However it is to be understood that the invention is not limited to the precise embodiment described and illustrated above. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A soft tissue enlarging material consisting essentially of fine particles of a pH-response water-absorbing swelling polymer having an average particle size of from 15 μm to 40 μm wherein swelling of said particles is completed within 10 minutes after immersion in a 10 mM phosphate buffer physiological saline solution at 37° C. and having a pH of 7.

2. The soft tissue enlarging material according to claim 1, wherein said particles are swollen to a particle size of 2 to 5 times greater than an unswollen size of the particles, after immersion in a 10 mM phosphate buffer physiological saline solution at 37° C. and having a pH of 7.

3. The soft tissue enlarging material according to claim 1, wherein said material is employed under skin or below a mucous membrane.

4. A soft tissue enlarging material consisting essentially of fine particles of a pH-response water-absorbing swelling polymer having an average particle size of from 15 μm to 40 μm wherein swelling of said particles with a body fluid in a living body is completed within 10 minutes.

5. The soft tissue enlarging material according to claim 4, wherein said particles are swollen to a particle size of 2 to 5 times greater than an unswollen size of the particles, after immersion in a 10 mM phosphate buffer physiological saline solution at 37° C. and having a pH of 7.

6. The soft tissue enlarging material according to claim 4, wherein said material is employed under skin or below a mucous membrane.

7. A soft tissue enlarging material comprising fine particles of a pH-response water-absorbing swelling polymer having an average particle size of from 15 μm to 40 μm wherein swelling of said particles is completed within 10 minutes after immersion in a 10 mM phosphate buffer physiological saline solution at 37° C. and having a pH of 7.

8. The soft tissue enlarging material according to claim 7, wherein said particles are swollen to a particle size of 2 to 5 times greater than an unswollen size of the particles, after immersion in a 10 mM phosphate buffer physiological saline solution at 37° C. and having a pH of 7.

9. The soft tissue enlarging material according to claim 7, wherein said material is employed under skin or below a mucous membrane.

10. A soft tissue enlarging material comprising fine particles of a pH-response water-absorbing swelling polymer having an average particle size of from 15 μm to 40 μm wherein swelling of said particles with a body fluid in a living body is completed within 10 minutes.

11. The soft tissue enlarging material according to claim 10, wherein said particles are swollen to a particle size of 2 to 5 times greater than an unswollen size of the particles, after immersion in a 10 mM phosphate buffer physiological saline solution at 37° C. and having a pH of 7.

12. The soft tissue enlarging material according to claim 10, wherein said material is employed under skin or below a mucous membrane.
